(19) United States
(12) Patent Application Publication
(10) Pub. No.: US 2025/0343801 A1
(43) Pub. Date: Nov. 6, 2025

(54) OPERATOR LEVEL AND ROLE-SPECIFIC AUTHORIZATION FOR OPERATING AN INFUSION PUMP

(71) Applicant: B. Braun Melsungen AG, Melsungen (DE)

(72) Inventors: Maria Buerger, Malsfeld (DE); Stefan Holz, Rotenburg (DE)

(21) Appl. No.: 19/192,584

(22) Filed: Apr. 29, 2025

(30) Foreign Application Priority Data
May 6, 2024 (EP) 24174354.1

(51) Int. Cl. H04L 9/40; A61M 5/142; G16H 40/67

(52) U.S. Cl. CPC H04L 63/105; A61M 5/142; G16H 40/67; H04L 63/0823; H04L 63/0861

(57) ABSTRACT

A protection system of or for a medical device is configured to issue identity-specific, selectable authorization releases to operators for carrying out authorization-dependent manipulations on the medical device. The system includes a data memory containing data that identifies a number of authenticatable operators to whom individual authorization levels are assigned or can be assigned, and an authentication device for this purpose. The data memory also includes the identity of a current operator and a validation device configured to confirm the identity of the authenticated operator based on the data stored in the data memory. An authorization release selection device is configured to select and grant a predetermined authorization release in the event of successful authentication, depending on the authorization level of the authenticated operator. A method can be performed for issuing identity-specific authorization releases to operators for executing authorization-dependent manipulations on a medical device.

### CROSS-REFERENCE TO RELATED APPLICATION

This application claims priority under 35 U.S.C. § 119 to European Application No. 24174354.1, filed on May 6, 2024, the content of which is incorporated by reference herein in its entirety.

### FIELD

The present disclosure relates to a protection system, in particular an electronic access protection system of or for a medical device (infusion pump, blood treatment machine, and/or similar intensive care equipment), as well as an (automated) method for granting an authorization to a specific user/user type for a specific medical device/device type.

### BACKGROUND

In order to be able to carry out sensitive (critical) manipulations/settings on a device, proof of authorization is usually required, which must be provided in an appropriate manner as described in the following example.

Authentication is generally understood to mean the process of checking a proof of identity for its authenticity. In the example of an operating system of a sensitive device that can grant access to a secure area, for example to adjust or manipulate the device, an operator first asserts their access authorization by presenting/reading a previously defined authentication medium or a corresponding proof of identification (chip card) or by entering an access code. The operating system then identifies the operator (user) or the operator type based on the means of authentication and then carries out the authentication, i.e., the verification of the assertion made about the authenticity. In the case of a code entry, this is done according to the principle of a combination lock, whereas in the case of proof of identification, its basic authenticity is first verified and then its correspondence with a stored pattern. Only when this verification is successful is the user granted access authorization, usually for the duration of a session or a specified period of time.

When using medical devices, it is also important that certain manipulations (functions/activities or settings) of the medical device are protected. This is necessary to ensure that unauthorized operators or strangers do not have access to specific manipulations, including settings and functions of the medical device. One of the reasons for this safeguard is that, in the event of misuse or willful interference, damage to the medical device, and/or harm to a person undergoing therapy with the medical device may occur.

It is currently known that protection against manipulation of medical devices such as these, including infusion pumps and blood treatment machines, is provided by a code lock for safety-related functions, such as the provision or supply of critical drugs. The code used for this is usually hard-coded by the manufacturer in the software of the medical device, i.e., permanently programmed into it. Currently, the definition of such a code, for example whether a drug supply is protected against certain manipulations, is carried out with the help of certain service tools, such as a drug and/or therapy database. A number of critical drugs (e.g., painkillers) and/or a number of critical treatment therapies are stored in such a service tool, which require the use of a code lock for the medical device.

If a critical drug is administered by means of the medical device or a critical treatment therapy is carried out by means of the medical device, the input of the permanently programmed release code is necessary in order to be able to carry out certain settings on the medical device during the ongoing therapy.

Usually, different groups of operators (user types) have different levels of access to medical devices, i.e., there is usually a user group that is only allowed to perform free manipulations and a user group that can also activate code-secured manipulations. In other words, in the case of non-critical drugs and/or therapies to be administered, device manipulations are often activated, whereas in the case of critical drugs and/or therapies, the permanently programmed code lock is used, so that only a few selected manipulations remain activated and a code must be entered for other manipulations.

However, this protection system is disadvantageous because the number of manipulations is determined by the hard coding of the code, thus restricting or even preventing the adaptation and/or extension of the code-specific manipulations. In other words, the only option with current medical devices is to use the programmed code to enable or disable all sensitive functions/manipulations (those relevant to patient safety).

### SUMMARY

The underlying purpose of this disclosure is to eliminate or at least improve the disadvantages described above. A preferred objective of the present disclosure is to provide a medical (access) protection system that offers greater flexibility with regard to the granting of access authorization.

The core idea of the present disclosure is to first establish or provide a (first) database in which a number (plurality) of different operator types or groups can be or are (flexibly) entered, i.e., a plurality of different hierarchy levels can be stored or are stored, wherein each hierarchy level is or can be assigned a specific or determinable authorization level or each hierarchy level corresponds to a specific or determinable authorization level. Behind each level of authorization is a number of selected or selectable manipulations or manipulation sets (manipulation lines) that are (at least partially) different from one another. This means that each authorization level can be assigned a predefined or individually (freely) compiled selection of manipulations, wherein the assigned manipulations can differ at least to some extent from one authorization level to the next. Furthermore, it is intended to establish or provide a further (second) database in which a number (majority) of different drugs and/or therapies can be or are (flexibly) entered, i.e., a number of different drugs and/or therapies can be stored or have been stored, to each of which one or more manipulations intended for release or entire degrees of authorization (comprising the associated manipulations) are or can be assigned.

Finally, for the device-side confirmation/verification, the operator must provide a proof of their hierarchy level in the form of a respective proof of identification (chip card, ID card, etc.) or a code in accordance with the state of the art mentioned at the beginning, to be provided by the operating personnel in order to obtain approval for those manipulations in accordance with the respective person-assigned authorization level (hierarchy level) and in accordance with the respective drug and/or therapy.

The present disclosure relates to an (access) protection system of or for a medical device, in particular an infusion pump, for providing/allocating drug-and/or therapy-specific selectable authorization releases to operators or groups of operators for executing authorization-dependent manipulations on the medical device. The protection system includes an initial data memory or data set (internal or external to the device) that contains data or into which data can be entered that identifies a plurality of user groups or hierarchy levels to which different degrees of authorization are or can be assigned, wherein each degree of authorization stands for specific or specifiable manipulations of the medical device. Furthermore, the protection system includes a second data memory or data set (internal or external to the device) that contains data or into which data can be entered that identifies a plurality of different drugs and/or therapies, to each of which specific or specifiable authorizations or at least one of the different degrees of authorization is or can be assigned in accordance with the first database/first data set. Finally, the protection system includes an authorization release selection device that is designed and configured to assign at least one of the hierarchy levels from the first data memory/data record and to enable those manipulations contained therein which match the drug-/therapy-related manipulations according to the second data memory/data record.

The following devices are preferred for providing proof of authorization:
an authentication device adapted to determine the identity of a current operator and
a validation device that is designed to confirm the identity of the authenticated operator on the basis of data stored in the data memory.

In other words, the (medical) electronic protection system allows a medical device to be manipulated. For this purpose, an authentication device (e.g., a card reader, scanner, input field for entering a code, etc.) is provided, which an operator uses for their identification or to prove their identity. The operator, so to speak, proves their operating data/user data/identity on this device. Further, preferably, this proof or identification is then checked for correctness using a validation device. The authorization release selection device is then set up to decide, based on the data (stored in advance) in the data stores, which manipulations on the medical device for the respective drug to be administered or therapy to be performed are released for the respective operator of the corresponding hierarchy level and which manipulations are not released. In other words, the system has data storage devices or can access such data storage devices in which persons or groups of persons/hierarchy levels, including manipulations assigned to hierarchy levels, are stored in a (first) data set and a number of individual manipulations or a number of different manipulation combinations are stored in another (second) data set, which are assigned to various drugs and/or therapies in a selected manner, wherein the authorization release selection device assigns certain manipulations or a manipulation compilation to an authenticated person in accordance with the hierarchy level assigned to that person. Depending on which drug is currently being administered or which therapy is being carried out, the hierarchy level assigned to the person concerned, according to the first data set, is then sufficient to release all the manipulations assigned to this hierarchy level (according to the first data record) or to only release some manipulations at this hierarchy level (according to the first data record) that match the manipulations assigned to the current drug or therapy (according to the second data record).

The advantage of the disclosure is that identity-specific authorizations can be or are granted, whereby different operating personnel have different authorization releases, which in turn are assigned to different drugs and/or therapies. In this way, it is possible to flexibly enable operators of a certain hierarchy level for the associated manipulations for some drugs and/or therapies partially or completely and to block them partially or completely for other drugs and/or therapies. It is also possible to change these assignments as desired or to expand them by adding further hierarchy levels and their assignments to drugs and/or therapies.

It has been shown that the disclosure as described above can achieve a high level of patient safety while also being very user-friendly.

Preferably, the medical device is an infusion pump.

Preferably, a single data memory/a single database can be divided into the first and second data memory or contain the first and second data set, wherein the hierarchy levels/the authorization levels with the respectively assigned manipulations are stored or can be stored in the first data set and the drugs and/or therapies with correspondingly assigned manipulations to be released or entire authorization levels are stored or can be stored in the second data set. This makes it possible to decouple the hard-coded manufacturer's input in the software code. Overall, it is possible to decide which manipulations are identity-specific and drug-/therapy-related, regardless of the manufacturer.

The two data sets can be programmed on the medical device itself or they can be programmed externally to the medical device on a separate device, for example a computer. When programming on a computer that is separate from the medical device, it is advantageous if the data sets are subsequently transferred to the data memory/memories. This transmission can be done using a cable connection or, alternatively, using a radio transmission, which can be part of the protection system. Exemplary radio transmissions are possible via Wi-Fi, Bluetooth, or NFC. The advantage of programming on an external computer is that a third person who is familiar with and responsible for defining the hierarchy/authorization levels can define these data records independently of the location of the medical device.

Ideally, after a successful authentication, the hierarchy/authorization level assigned to this person is compared with the hierarchy/authorization level stored in the first data record, in order to issue the authorization release if these two levels match. If there is no match between the respective hierarchy/authorization levels, no authorization is granted and the manipulation(s) is/are blocked.

Preferably, the hierarchy/authorization level of the operator can have a variable X. This value X can preferably have one of two different values, for example, a binary code with the value “1” or “0”, or a truth value with the value “true” or “false”. Alternatively, all other values are also conceivable for a two-stage differentiation. Alternatively, the variable X can have an n-different number of values, so that a gradation of the authorization level is given. n can be a positive, finite number.

Preferably, the hierarchy/authorization level according to the first data record can have a variable Y. This variable Y can preferably have one of two different values. For example, a binary code with the value “1” or “0”, or a truth value with the value “true” or “false”. Alternatively, all other values are also conceivable for distinguishing between a two-stage demarcation. Further alternatively, variable Y can have an n-different number of values, so that a gradation of the authorization level is given. n can be a positive, finite number.

It is advantageous if, after a successful authentication, the authorization level of the operator, in this case variable X, is compared with the authorization level according to the first data record, in this case variable Y, in order to grant authorization if these levels match. This comparison makes it possible to uniquely verify authorization or authorization, which ensures that the operator only receives approval for the manipulation assigned to him/her, depending on the drug and therapy.

It is advantageous if an identity code is (additionally) assigned to an operator in the first data record. The operator can be clearly identified using the identity code. Preferably, the identifier is a numeric or alphanumeric code.

Preferably, the authenticatable operator can be assigned to an operator group or be assignable. One advantage of an operator group is that a large number of operators can be assigned or are assigned to this group. This means that fewer individual operators with respective authorization levels need to be defined. The individual operating groups can include, for example, nursing staff, the doctor on the ward, the pain team, the ward supervisor, etc. The operating groups can thus represent different hierarchy levels overall. In principle, these individual operating groups can represent different qualifications. Another advantage of this subdivision is that only specific manipulations are or can be defined for each of these operating groups.

The first data set and the second data set can each be stored as a data matrix. An example of how the data matrix should be designed for the first data set can be found in Table 1 below.

Example of a release concept

TABLE 1
Release matrix

| | Hierarchy level | | | | |
|---|---|---|---|---|---|
| | Doctor on | Pain | Ward | Nursing | |
| | the ward | team | supervision | staff | |
| | Code: | Code: | Code: | Code: | (more |
| Protected function | 8657 | 5481 | 3251 | 1567 | . . . ) |

| | | | | |
|---|---|---|---|---|
| Change flow rate | X | X | | |
| Change to disposable | X | X | X | X |
| items | | | | |

However, the present revelation is not limited to the four exemplary hierarchy/authorization levels and the two manipulations. Overall, a large number of hierarchy/authorization levels and a large number of manipulations can be defined.

An example of how the data matrix should be designed for the second data set can be found in Table 2 below.

However, the present revelation is not limited to the two exemplary manipulations and the two exemplary drugs/therapies. Overall, a wide range of manipulations and drugs/therapies can be defined.

Preferably, an authentication medium/key is assigned to an operator or group of operators.

Preferably, at least one means of authentication is assigned to the identifier. This makes it possible to identify the operator during authentication.

Preferably, an authentication means for demonstrating an identity has a numeric code or an alphanumeric code or a biometric identifier or an RFID identifier. The numeric code and/or the alphanumeric code can be a password defined by an operator, but alternatively it can also be a characteristic assigned to the operator, for example a specific code, in particular a personnel number. A biometric identifier can be a fingerprint, a face scan, or an iris scan.

The data memory can be arranged as an internal memory in the medical device or can be designed as an external memory that is connected to the medical device.

The authentication device can be arranged as an internal device in the medical device or can be designed as an external device that is connected to the medical device.

The authentication device may comprise a reader for scanning the authentication means or an input device for entering the authentication means or a data receiver, for example, an RFID receiver or NFC receiver. The input device can be an (external) keyboard or control buttons on the authentication device or it can be a touchscreen.

The validation device can be arranged as an internal device in the medical device or designed as an external device that is connected to the medical device.

The authorization release selection device can be arranged as an internal device in the medical device or can be designed as an external device that is connected to the medical device.

It is advantageous if the authentication device and the validation device are connected to one another by means of a (system-internal) data communication line and/or the authorization release selection device and the validation device are connected to one another by means of a (system-internal) data communication line and/or the authorization release selection device and the authentication device are connected to one another by means of a (system-internal) data communication line. Alternatively, the authentication device and the validation device may be connected to each other by means of a wireless communication link and/or the authorization release selection device and the validation device may be connected to each other by means of a wireless communication link and/or the authorization release selection device and the authentication device may be connected to each other by means of a wireless communication link.

It is advantageous if the data memory is connected to the authentication device and/or the validation device and/or the authorization release selection device by means of a (system-internal) data communication line or a wireless communication link.

The wireless communication connection can be implemented using Wi-Fi, Bluetooth, or NFC.

Preferably, the medical device may be connected to the data storage device and/or the authentication device and/or the validation device and/or the authorization release selection device by means (respectively) of a data communication line (internal to the system) or a wireless communication link. In other words, the medical device can be designed to function simultaneously as an authentication device, a validation device, and/or an authorization release selection device. This creates a particularly compact protection system.

In the case of a purely system-internal data communication line for communication between the devices and the data storage device as well as with the medical device, the protection system can be designed as an isolated solution. An isolated application has the advantage in this case that the protection system works independently. This protects the protection system from external influences/interference in the form of hacker attacks, for example.

The validation device can be a server unit that communicates wirelessly with the other devices and the medical device.

The disclosure also relates to a method for granting identity-specific authorizations to operators for performing authorization-dependent manipulations on a medical device.

First, data is entered into a first data store or data set, which identifies a plurality of operator groups or hierarchy levels, to which different degrees of authorization are assigned, wherein each degree of authorization is assigned selectable manipulations of the medical device. Furthermore, data identifying a plurality of different drugs and/or therapies are entered in a second data store or data set, to each of which selectable manipulations to be activated or at least one of the different degrees of authorization with respect to one another are assigned in accordance with the first database/first data set. Finally, at least one of the hierarchy levels from the first data store/data record is assigned to an operator on the basis of the credentials provided by that operator, and those manipulations contained therein that match the drug-/therapy-related manipulations according to the second data store/data record are activated.

Preferably, to provide proof of authorization, an authentication method is first provided at an authentication device to verify the identity of the operator. The authentication device then recognizes the identification data transmitted by the operator. The data is then transferred to a validation device to verify the data. Then the authentication to confirm the identity is carried out on the basis of the number of operators or operator groups/hierarchy levels stored in a data memory. Finally, an authorization release is issued by an authorization release selection device after successful authentication, depending on the hierarchy/authorization level of the authenticated operator and the drug-/therapy-related manipulations.

It is advantageous if the authorization release selection device for the authorization release compares the authorization levels of the first data record with the authorization level assigned to the operator and, if the authorization levels match, grants the authorization release.

### BRIEF DESCRIPTION OF THE DRAWINGS

The disclosure will be explained in more detail below with reference to a preferred exemplary embodiment, with reference to the accompanying figures.

FIG. 1 is a schematic view of a protection system in a first embodiment;

FIG. 2 is a schematic view of the protection system in a second embodiment;

FIG. 3 is a schematic view of the protection system in a third embodiment; and

FIG. 4 is a flowchart of the authorization approval process.

### DETAILED DESCRIPTION

The following describes exemplary embodiments of the present disclosure on the basis of the associated figures.

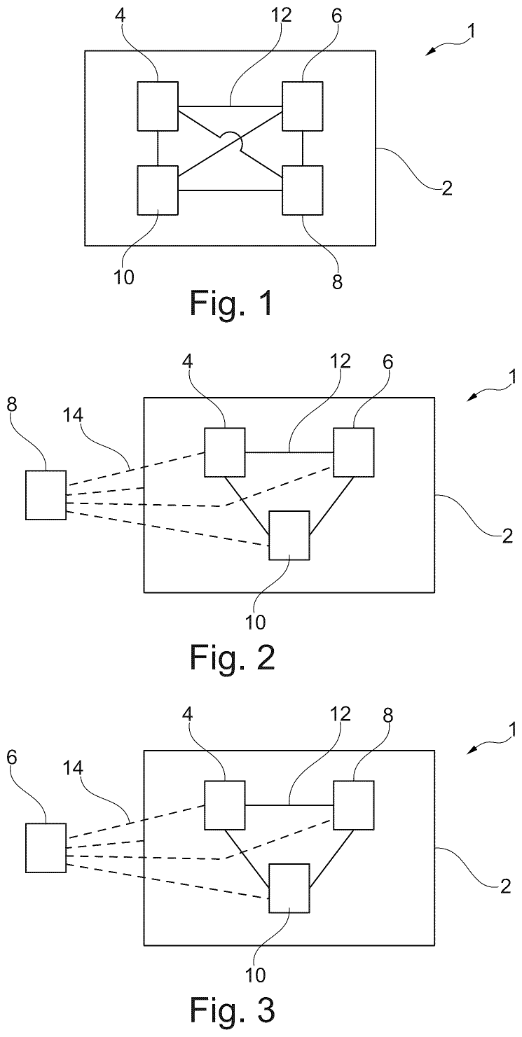

FIG. 1 shows a schematic view of an electronic access protection system 1 in a first embodiment. The protection system 1 is shown here for a medical device, in particular an infusion pump 2. The protection system 1 is designed to issue identity-specific selectable authorizations to operators for performing authorization-and drug-/therapy-dependent manipulations on the medical device 2.

The protection system 1 has at least one data memory 4 for this purpose. The data store 4 includes a first data set containing data regarding a plurality of hierarchy/authorization levels, to which certain manipulations are assigned in each case. The manipulations can differ at least partially under the respective hierarchy/authorization levels, they can partially be the same, or the number of manipulations can differ under the respective hierarchy/authorization levels. The data memory 4 comprises a second data set containing data relating to a plurality of drugs and/or therapies, to each of which certain manipulations to be unlocked are assigned. The manipulations to be unlocked can differ at least partially among the respective drugs/therapies, can partially be the same, or the number of manipulations can differ among the respective drugs/therapies.

Furthermore, the protection system 1 has an authentication device 6. The authentication device 6 is designed to verify the identity of the current operator.

Furthermore, protection system 1 has a validation device 8. The validation device 8 is designed to confirm the identity of the authenticated operator on the basis of the data stored in the data memory 4.

The protection system 1 finally comprises an authorization release selection device 10. This person is designated and trained to select and issue a (pre-) determined authorization clearance based on the hierarchy/authorization level of the authenticated operator in the event of a successful authentication. In the context of this authorization release, the operator in question is given access to those manipulations that are included in the assigned hierarchy/authorization level according to the first data record and that match the manipulations to be released for the current drug or therapy according to the second data record.

In the present first embodiment, the protection system 1 is designed as an isolated solution. The authentication device 6, the validation device 8, the data memory 4 and the authorization release selection device 10 are formed/arranged internally in the medical device 2. The devices 6, 8, 10 and the data memory 4 are interconnected/coupled to one another by means of data transmission lines 12. This means that data used for the authorization release can be exchanged.

FIG. 2 shows the protection system 1 in a different embodiment to that shown in FIG. 1, a second embodiment. In the present case, the validation device 8 is designed/arranged externally to the medical device 2. Instead of an internal data transmission line 12 as shown in FIG. 1, the data transfer of the validation device 8 takes place via a radio connection 14. Thus, the validation device is connected to the medical device 2 and the other devices 6, 10 and the data storage 4 by means of the radio connection 14.

FIG. 3 shows the protection system 1 in a third embodiment. In this case, the authentication device 6 is designed as a separate device from the medical device 2. The authentication device 6 is connected to the medical device 2 and the other devices 8, 10 and the data storage 4 by means of a radio connection 14.

FIGS. 1 to 3 show different exemplary embodiments. However, the disclosed protection system 1 is not limited to these embodiments. The data memory 4 and/or the authorization release selection device 10 can also be designed externally to the medical device 2, as shown in the versions in FIGS. 1 to 3, and can optionally have a radio connection 14 or a cable connection.

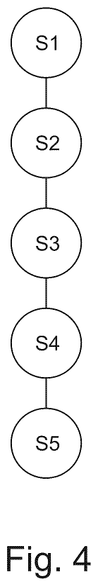

FIG. 4 shows a flowchart of the authorization approval process.

In the first step (S1), an authentication means is provided at the authentication device 6 to establish an identity, in this case the identity of the operator.

In a second step (S2), the authentication device 6 recognizes the authentication means and determines the operator on the basis of the data stored in the data memory 4.

In a third step (S3), the data of the identity is transmitted from the authentication device 6 to the validation device 8 in order to carry out an authentication, in this case a verification of the data, ergo of the operator.

The authentication takes place in a fourth step (S4).

If the authentication of the operator is successful, a fifth step (S5) is carried out in which an authorization level of the operator is compared with an authorization level of the manipulation using the authorization release selection device 10. If the authorization level of the operator and the authorization level of the manipulation match, an authorization release is granted. If the authorization level of the operator and the authorization level of the manipulation do not match, no authorization clearance is granted.

### LIST OF REFERENCE SIGNS

1 Protection system
2 Medical device
4 Data storage
6 Authentication device
8 Validation device
10 Authorization release selection device
12 Internal data transmission line
14 Radio communication

1. A protection system of or for a medical device for granting authorization releases to operators for carrying out authorization-dependent manipulations on the medical device, the protection system comprising:
at least one data memory in which a first data record is created, which contains data which identifies a plurality of hierarchy or authorization levels, to each of which specific or selectable manipulations are assigned or can be assigned, and in which a second data record is created which contains data which identifies a plurality of drugs and/or therapies, to which manipulations to be enabled are assigned or can be assigned in each case; and
an authorization release selection device which is configured to assign at least one of the plurality of hierarchy or authorization levels to an operator as a function of an authorization certificate of the operator, and to enable only those manipulations assigned to the at least one of the plurality of hierarchy or authorization levels according to the first data record that correspond to the manipulations to be enabled according to the second data record that are assigned to a currently used drug or a currently applied therapy.

2. The protection system according to claim 1, further comprising:
an authentication device that is configured to determine an identity of the operator; and
a validation device that is configured to confirm the identity of the operator based on the data stored in the at least one data memory.

3. The protection system according to claim 2, further comprising an authenticating identifier configured to be assigned to the operator.

4. The protection system according to claim 3, wherein the authenticating identifier comprises a numeric code or an alphanumeric code.

5. The protection system according to claim 3, wherein the authenticating identifier comprises a biometric identifier.

6. The protection system according to claim 5, wherein the authenticating identifier comprises an RFID identifier.

7. The protection system according to claim 5, wherein the authentication device comprises a reader for scanning the authenticating identifier.

8. The protection system according to claim 5, wherein the authentication device comprises an input device for manually entering the authenticating identifier.

9. The protection system according to claim 5, wherein the authentication device comprises a data receiver.

10. The protection system according to claim 3, wherein the authentication device and the validation device are connected to one another by a system-internal data communication line.

11. The protection system according to claim 3, wherein the authorization release selection device and the validation device are connected to each other by a system-internal data communication line.

12. The protection system according to claim 1, wherein the medical device is an intensive-care device.

13. The protection system according to claim 12, wherein the intensive-care device is an infusion pump.

14. The protection system according to claim 12, wherein the intensive-care device is a blood treatment machine.

15. A method for granting identity-specific authorizations to operators for performing authorization-dependent manipulations on a medical device, the method comprising the steps of:
entering data into a first data store or data set, wherein the data identifies a plurality of operator groups or hierarchy levels to which different authorization levels are assigned, wherein each authorization level is assigned selectable or selected manipulations of the medical device;
entering data into a second data store or data set, wherein the data identifies a plurality of different drugs and/or therapies, to each of which selectable or selected manipulations to be activated are assigned or at least one of the different authorization levels in accordance with the first data store or data set; and
assigning at least one of the hierarchy levels from the first data store or data set to an operator depending on credentials provided by the operator, and enabling manipulations contained in the at least one of the hierarchy levels that match the selectable or selected manipulations according to the second data store or data set.
